(19) United States
(12) Patent Application Publication
(10) Pub. No.: US 2023/0022804 A1
(43) Pub. Date: Jan. 26, 2023

(54) AN INDIVIDUALLY PACKED SANITARY ARTICLE

(71) Applicant: Essity Hygiene and Health Aktiebolag, Goteborg (SE)

(72) Inventor: Charlotte Pollard, Asa (SE)

(21) Appl. No.: 17/783,533

(22) PCT Filed: Dec. 13, 2019
(86) PCT No.: PCT/SE2019/051281

§ 371 Date: Jun. 8, 2022

(51) Int. Cl. A61F 13/551; A61F 13/472; A61F 13/56

(52) U.S. Cl. CPC A61F 13/5514; A61F 13/47245; A61F 13/5616

(57) ABSTRACT

The present disclosure relates to a folded sanitary article (100,200), such as a sanitary napkin or a pantiliner T the sanitary article comprises a topsheet (102,202), a backsheet (103,203) and an absorbent core (104,204) arranged between the topsheet (102,202) and the backsheet (103,203). The sanitary article (100,200) extends in a longitudinal direction (L) and in a transverse direction (T). The sanitary article (100,200) has a first longitudinal side edge (105,205), a second longitudinal side edge (106,206), a front edge (107,207) and a rear edge (108,208). The sanitary article (100,200) has a first fastening wing (109,209) extending at the first longitudinal side edge (105,205) and a second fastening wing (110,210) extending at the second longitudinal side edge (106,206). The first and second fastening wings (109,110,209,210) are asymmetrical with respect to each other, as seen along any longitudinal axis extending in the longitudinal direction (L). The absorbent core (104,204) comprises an absorbent core front edge (111,211), an absorbent core rear edge (112,212), an absorbent core first longitudinal side edge (113,213) and an absorbent core second longitudinal side edge (114,214), the absorbent core (104) having an absorbent core width (Wabs) and a maximum absorbent core width (wabsMAx). The first and the second fastening wings (109,110) of the sanitary article (100,200) are folded onto the topsheet (102,202) of the sanitary article (100,200). The folded sanitary article (100,200) has a folded article width (Wf) and the folded article width (wrs) is not more than 10 mm greater than the maximum absorbent core width (WabsMAx). The present disclosure also relates to a package including a plurality of folded sanitary articles and an array of folded sanitary articles.

### CROSS REFERENCE TO RELATED APPLICATIONS

This application is a National Stage application of PCT/SE2019/051281, filed Dec. 13, 2019, which is incorporated by reference in its entirety herein.

### TECHNICAL FIELD

The present disclosure pertains to a sanitary article such as a sanitary napkin or a pantiliner. In particular, this disclosure pertains to a sanitary article having asymmetrical fastening wings being folded. This disclosure also relates to a folded sanitary article individually packed in a wrapping sheet.

### BACKGROUND

A sanitary article of the kind to which this disclosure relates includes an absorbent pad arranged between a topsheet and a backsheet, it further includes so-called fastening wings provided with adhesive regions for fastening of the wings in a user undergarment. When using a sanitary article, the article should be designed to be comfortable to wear for the user and it should provide a high level of protection for the user undergarment and clothing. When wearing the sanitary article, the user often moves around which may cause the sanitary article to move as well. This may result in incorrect positioning of the sanitary article and undesired wrinkling and folding of the sanitary article, which may negatively affect comfort, absorption and protection of the user garments. To secure that the sanitary article remains in place, the size and width of the fastening wings may be increased.

Sanitary articles may be individually packed in primary packages in the form of wrapping sheets. The user may use the wrapping sheets for disposal of used sanitary articles. When bringing along separate sanitary articles in for example a handbag or toilet bag, the wrapping sheet protects the sanitary article from being soiled and individually packed sanitary articles are therefore more hygienic to bring along. The wrapping sheet is however an additional amount of material, which has a negative impact both in economic and environmental terms. Therefore, there is a strong desire to reduce the environmental impact and reduce the costs of sanitary articles. A very large number of sanitary articles are constantly being produced and every saving for each article represents a large total saving.

It is an object of the present invention to reduce the environmental impact of sanitary articles while yet providing a comfortable, secure and hygienic sanitary article.

### SUMMARY

One or more of the above objects is achieved with a folded sanitary article in accordance with claim 1, a package for a plurality of sanitary articles according to claim 19 and an array of folded sanitary articles according to claim 20.

Further advantages and advantageous features of the invention are disclosed in the following description and in the dependent claims.

According to a first aspect, the present disclosure relates to a folded sanitary article, such as a sanitary napkin or a pantiliner. The sanitary article comprises a topsheet, a backsheet and an absorbent core arranged between the topsheet and the backsheet. The sanitary article extends in a longitudinal direction and in a transverse direction. The sanitary article has a first longitudinal side edge and a second longitudinal side edge, a front edge and a rear edge. The sanitary article has a first fastening wing extending outboard at the first longitudinal side edge and a second fastening wing extending outboard at the second longitudinal side edge. The absorbent core comprises an absorbent core front edge, an absorbent core rear edge and absorbent core first and second longitudinal edges. The absorbent core has a maximum absorbent core width. The first and the second fastening wings of the sanitary article are folded onto the topsheet of the sanitary article. The folded sanitary article has a folded article width. The first and second fastening wings are asymmetrical with respect to each other, as seen along any longitudinal axis extending in the longitudinal direction and the folded article width is not more than 10 mm greater than the maximum absorbent core width.

Hence, the first and the second fastening wings are asymmetrical with respect to a sanitary article longitudinal centerline.

The first and second fastening wings may be asymmetrical with respect to any transverse axis. The first and second fastening wings may thus be asymmetrical with respect to a fastening wing transverse centerline.

The first and the second fastening wing may be symmetrically disposed with respect to each other along a diagonal axis extending in a direction in the plane of the sanitary article, and with an angle within the range of from 10° to 65°, such as within the range of from 30° to 55°, with respect to the longitudinal centerline of the sanitary article. Hence, the first and the second fastening wings may be mirror-images of each other if being turned 180° and may be arranged to extend outboard at the respective first and second longitudinal side edge at a respective location along the respective first and second longitudinal side edge such that they are symmetrically disposed with respect to each other.

The fact that fastening wings are symmetrically disposed with respect to each other along a diagonal axis extending in a direction in the plane of the sanitary article and with an angle within the range of from 10° to 65° with respect to the longitudinal centerline of the sanitary article, and asymmetrically with respect to each other as seen along any longitudinal axis extending in the longitudinal direction, may promote an improved fastening symmetry of the fastening wings with fastening wings having an increased width without overlapping each other upon fastening of the fastening wings to the undergarment. This has been seen to provide enhanced fastening over time by preventing twisting and turning of the sanitary article in the undergarments, which could lead to leakage and discomfort for the user.

The fastening of a sanitary napkin to an undergarment by means of fastening wings may be improved by means of larger fastening wings having a greater extension, as seen in the transverse direction. During manufacturing the fastening wings are folded on top of the topsheet, the fastening wings are subsequently provided with adhesive on a garment facing side thereof and then the adhesive zones are covered with release paper. To avoid that the adhesive zones interfere with each other resulting in that the fastening wings stick together upon folding, the fastening wings should not be folded to overlap each other. If wider fastening wings are desired, the fastening wings could of course be folded such that a respective fold line is provided with a greater distance to the absorbent core respective longitudinal side edge. However, this increases the width of the folded article and larger sized wrapping sheet and/or packages for such sanitary articles are desirable. This is undesirable both in environmental and in economic aspects due to the increased material consumption and increased packaging sizes during storage and transport. A small decrease in folded size for each article may lead to a relatively large total decrease in material consumption and space consumed during storage and transports. This is due to the great numbers of sanitary article constantly being manufactured, stored and transported.

By giving the fastening wings an asymmetrical shape according to the present disclosure it has been found by the present inventors that the extension of the fastening wings in the transverse direction may be increased without having the problems of the fastening wings overlapping each other, a wrapping sheet increased size and large packages.

The maximum absorbent core width is measured in the transverse direction and at the widest portion of the absorbent core along a length of the absorbent core.

The width of the absorbent core corresponds to the width of the widest absorbent core layer arranged between the topsheet and the backsheet. The width may, when the fastening wings are folded on top of the topsheet of the sanitary article, for example be measured at the backsheet side. Individual fibers sticking out from the outer contour of the absorbent core should not be considered when measuring the width.[0017] With “folded article width” herein is meant the width of the final folded article at the widest location as seen along a length of the folded sanitary article and in the transverse direction.

The absorbent core according to the present disclosure may include one or more absorbent layers. The sanitary article as disclosed herein may include an acquisition layer, arranged between the topsheet and underlying absorbent layers for receiving and spreading liquid to the underlying absorbent layers.

Different types of material layers may be used as an acquisition layer. Acquisition layers may be soft and compliant and may therefore be easy to fold around the longitudinal side edges of the absorbent core together with the topsheet and the backsheet without influencing the size of the folded sanitary article. Such soft and compliant materials include a spunlace nonwoven, an apertured film, an apertured nonwoven, a laminate of an apertured film and nonwoven, a foam material and a highloft nonwoven. In the context of the present disclosure, when a spunlace nonwoven, an apertured film, an apertured nonwoven, an apertured film and nonwoven laminate, a foam material or a highloft nonwoven is included as an acquisition layer, the acquisition layer is not considered to form part of the absorbent core for measuring the width of the absorbent core.

A high-loft nonwoven is a carded, air through bonded nonwoven made of staple fibers with a gsm between 20-150 gsm, such as 30-80 gsm. The staple fibers are 2D crimped or 3D spiral shaped. The fibers are typically polyester, polyethylene, polypropylene and/or polylactic acid fibers, the fibers may be bicomponent fibers. The fibers are treated to become hydrophilic.

Material layers having a larger stiffness and a higher volume may alternatively be used for spreading liquid into the underlying absorbent core, such layers are not easy to fold together with the topsheet and the backsheet along the longitudinal edge portions, such stiffer layer include airlaid nonwoven. In the context of the present disclosure, when an airlaid nonwoven material is used as an acquisition layer, the acquisition layer is considered to form part of the absorbent core for measuring the width of the absorbent core.

The term “sanitary article” refers to products that are placed against the skin of the wearer to absorb and contain body exudates, such as menstrual fluid and urine. The disclosure mainly refers to disposable sanitary articles, which means articles that are not intended to be laundered or otherwise restored or reused as a sanitary article. Examples of disposable sanitary articles include feminine sanitary napkins, pantiliners and the like.

The first and second fastening wings may be folded around the absorbent core with the respective fastening wing fold line being arranged not more than 8 mm, or 6 mm, or 4 mm, such as 3 mm, or 2 mm from the first and the second longitudinal edge of the absorbent core respectively. The first and the second fastening wing may be folded directly at and around the respective first and second longitudinal edge of the absorbent core.

The first fastening wing has may have a first fastening wing edge section and the second fastening wing may have a second fastening wing edge section. When the fastening wings are folded onto the topsheet of the sanitary article, the first and the second fastening wing edge sections are arranged opposite and adjacent each other, a distance between the first and the second fastening wing edge sections may be within the range of from 0 mm to 25 mm, optionally the distance between the first and the second fastening wing edge sections may be within the range of from 0 mm to 10 mm, optionally the distance between the first and the second fastening wing edge sections may be within the range of from 0 mm to 7 mm.

The first fastening wing edge section and the second fastening wing edge section may each have a length within the range of from 10 mm or more along the first and the second fastening wing lateral edge. The first fastening wing edge section and the second fastening wing edge section may each have a length within the range of from 15 mm to about 80 mm, as measured along a respective edge of the first and second fastening wing.

Each of the fastening wings may extend over a longitudinal centerline of the sanitary article when arranged in the folded configuration without the fastening wings overlapping each other.

A width of the first fastening wing may continuously increase over at least 50% of the first fastening wing, as seen in the longitudinal direction, from the rear portion and towards the front portion of the sanitary article, optionally over at least 75% of the first fastening wing, the width being measured in the transverse direction and from the first longitudinal absorbent core edge.

A width of the second fastening wing may continuously increase over at least 50% of the second fastening wing, as seen in the longitudinal direction, from the front portion and towards the rear portion of the sanitary article, optionally over at least 75% of the second fastening wing, the width being measured in the transverse direction and from the second longitudinal absorbent core edge.

The first fastening wing may have a first fastening wing maximum width and the second fastening wing may have a second fastening wing maximum width, each of the fastening wing widths being measured from the respective first and a second longitudinal absorbent core edge and at the widest section of the respective fastening wings as measured in the transverse direction, and wherein the sum of the first and the second fastening wing widths is more than 10 mm greater than the absorbent core width, as measured at a sanitary article transverse centerline

The folded sanitary article may be a tri-folded sanitary article. The sanitary article may be folded over a first and a second transverse folding lines, the sanitary article may alternatively be folded over one transverse folding line or over three transverse folding lines.

The absorbent core may have an essentially rectangular shape having an absorbent core width not varying more than ±20 mm along a length of the absorbent core.

The absorbent core may have an essentially rectangular shape having an absorbent core width not varying more than ±10 mm along a length of the absorbent core.

The absorbent core may have an essentially rectangular shape having an absorbent core width not varying more than ±5 mm along a length of the absorbent core.

The absorbent core may have longitudinal folding indications facilitating longitudinal folding of a non-rectangular shaped absorbent core to an essentially rectangular or square folded shape. When the absorbent core has such longitudinal folding indications, the sanitary article may be folded at an inner side of the respective first and second longitudinal side edges of at least parts of the absorbent core.

The absorbent core may an essentially rectangular or square folded shape.

Sanitary articles may have a wider front section and/or rear section and a narrower waist section. The absorbent core may follow the outer contour of the sanitary article and has a wider front and/or rear section and a narrower waist, or alternatively only the topsheet and backsheet have such shaped outer contour. As the topsheet and the backsheet are highly flexible layers, they are easily foldable along the longitudinal edges of the absorbent core to reduce the width of the folded sanitary article. A sanitary article having an absorbent core with a wider front section and/or rear section may render the folded article wider and the wrapping sheet material and packages larger, as it is the widest part of the folded article that sets the size requirement. The fact that the absorbent core has an essentially rectangular or square folded shape in combination with that the fastening wings may be folded closer to the absorbent core longitudinal edges, enables smaller size wrapping sheets and packages.

According to a second aspect of the present disclosure, a folded sanitary article according to the first aspect is individually packed in a wrapping sheet. The wrapping sheet has first and second transverse sheet edges and first and second longitudinal sheet edges, a sheet width extending between the first and the second sheet longitudinal edges. The wrapping sheet comprises a longitudinal sealing area sealing the respective first and second longitudinal edges of the wrapping sheet.

The sheet width may be maximum 60% greater than the width of the absorbent core, as measured at the transverse centerline.

The sheet width may be maximum 40% greater than the width of the absorbent core, as measured at the transverse centerline.

The unfolded width of the sanitary article prior to folding the first and the second fastening wings onto the topsheet, as measured at the sanitary article transverse centerline, may be maximum 22% greater than the width of the wrapping sheet.

A distance between the first longitudinal absorbent core edge and the first longitudinal sealing area of the wrapping sheet and a distance between the second longitudinal absorbent core edge and the second longitudinal sealing area of the wrapping sheet may each range from 0 mm to 50 mm, as seen along the entire length of the absorbent core.

A distance between the first longitudinal absorbent core edge and the first longitudinal sealing area of the wrapping sheet and a distance between the second longitudinal absorbent core edge and the second longitudinal sealing area of the wrapping sheet may each range from 5 mm to 25 mm, as seen along the entire length of the absorbent core.

A distance between the first longitudinal absorbent core edge and the first longitudinal sheet edge and a distance between the second longitudinal absorbent core edge and the second longitudinal sheet edge may each range from 0 mm to 35 mm.

A distance between the first longitudinal absorbent core edge and the first longitudinal sheet edge and a distance between the second longitudinal absorbent core edge and the second longitudinal sheet edge may each range from 5 mm to 30 mm.

A total surface area of the wrapping sheet may be maximum 150% greater than a total surface area of the absorbent core. A total surface area of the wrapping sheet may be maximum 120% greater than a total surface area of the absorbent core.

The total surface area of the wrapping sheet may be maximum 50% greater than a total surface area of the absorbent core.

The total surface area of the wrapping sheet may be maximum 40% greater than the total surface area of the absorbent core.

The sanitary article may be wrapped by a wrapping sheet having a wrapping sheet length being within the range of from 75% to 135% of a length of the sanitary article.

The wrapping sheet length may be within the range of from 75% to 100% of the length of the sanitary article, optionally the wrapping sheet length may be within the range of from 75% to 90% of the length of the sanitary article.

According to a third aspect of the present disclosure, the present disclosure relates to a package including a plurality of folded articles according to the first and/or second aspect of the present disclosure.

According to a fourth aspect, the present disclosure relates to an array of folded sanitary articles according to the first or second aspect. The array comprises a first folded sanitary article and a second folded sanitary article, wherein the first folded sanitary article has a first sanitary article length and the second folded sanitary article has a second folded sanitary article length and wherein the first sanitary article length is at least 20% longer than the second sanitary article length.

The sanitary article length being measured when the sanitary article is in an unfolded configuration.

The array of folded sanitary articles may comprise a third folded sanitary article and wherein the second sanitary article is at least 15% longer than the third sanitary article.

The first sanitary article, the second sanitary article and the third sanitary article in the array of folded sanitary articles according to the fourth aspect may be a sanitary article according to any one of the first or second aspect embodiments.

It has surprisingly been found that sanitary articles according to the present disclosure, despite difference in size, reduces the folded article width compared to the absorbent maximum core width in a consistent manner and that a reduced folded article width and the advantages associated with this reduction may be achieved for an array of sanitary articles having different lengths.

According to a fifth aspect, the present disclosure relates to an array of folded sanitary articles individually packed in a wrapping sheet according to the second aspect. The array comprises a first folded and individually packed sanitary article and a second folded and individually packed sanitary article. The wrapping sheet width being maximum 60% greater than the width of the absorbent core, as measured at a transverse centerline of the sanitary article in an unfolded configuration. The first folded and individually packed sanitary article having a first sanitary article length and the second folded and individually packed sanitary article has a second folded sanitary article length and wherein the first sanitary article length is at least 20% longer than the second sanitary article length

The first and second folded sanitary articles individually packed in a wrapping sheet in the array of folded sanitary articles individually packed in a wrapping sheet according to the fifth aspect may be a folded sanitary articles individually packed in a wrapping sheet according to any one of the second aspect embodiments.

It has surprisingly been found that an individually packed sanitary articles according to the present disclosure, despite difference in size, reduces both the folded article width compared to the absorbent maximum core width and the wrapping sheet width in a consistent manner and that a reduced folded article width and the advantages associated with this reduction may be achieved for an array of individually packed sanitary articles having different lengths.

### BRIEF DESCRIPTION OF THE DRAWINGS

The present invention will be further explained hereinafter by means of non-limiting examples and with reference to the appended drawings wherein:

FIG. 1 illustrates an unfolded sanitary article according to the present disclosure;

FIG. 2 illustrates an unfolded alternative sanitary article according to the present disclosure;

FIG. 3a-3f illustrate folding and wrapping of a sanitary article according to FIG. 1.

### DETAILED DESCRIPTION

The invention will be described more closely below by reference to an exemplary embodiment. The invention may however be embodied in many different forms and should not be construed as limited to the embodiments set forth in the drawings and the description thereto.

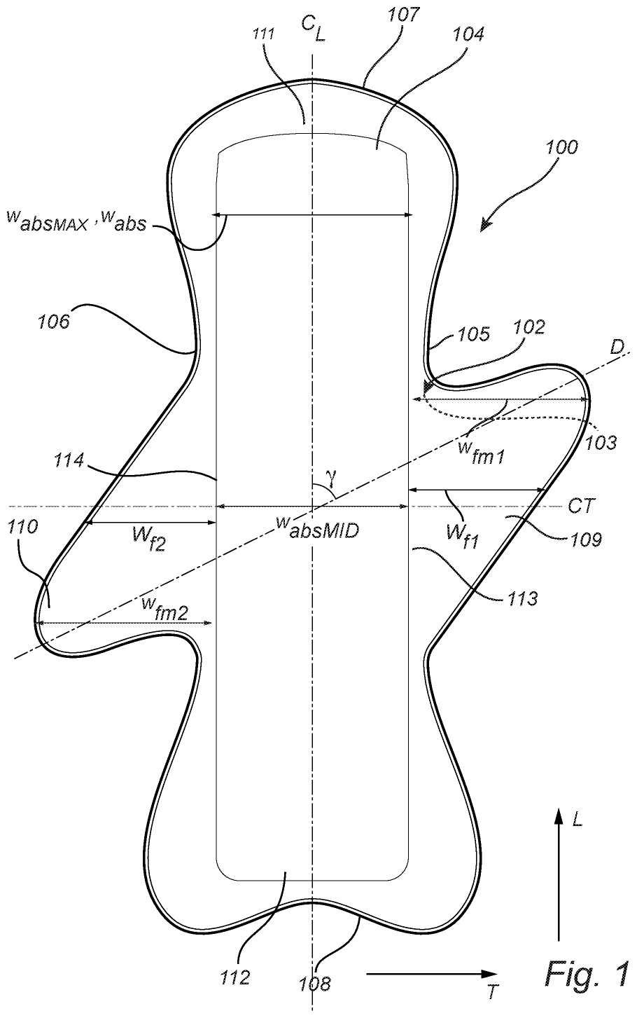

FIG. 1 illustrates a sanitary article 100 according to the present disclosure. The sanitary article comprising a topsheet 102, a backsheet 103 and an absorbent core 104 arranged between the topsheet 102 and the backsheet 103. The sanitary article 100 extends in a longitudinal direction L and in a transverse direction T. A longitudinal centerline C_{L} of the sanitary article 100 extends in the longitudinal direction L. The sanitary article 100 has a first longitudinal side edge 105 and a second longitudinal side edge 106, a front edge 107 and a rear edge 108. The sanitary article 100 has a first fastening wing 109 extending outboard at the first longitudinal side edge 105 and a second fastening wing 110 extending outboard at the second longitudinal side edge 106. The absorbent core 104 comprises an absorbent core front edge 111, an absorbent core rear edge 112, an absorbent core first longitudinal side edge 113 and a second longitudinal side edge 114. The absorbent core 104 has an absorbent core width wabs and a maximum absorbent core width w_{absMAX}. Since the absorbent core 104 depicted in this figure has an essentially rectangular shape, the absorbent core width wabs is equal to the maximum absorbent core width w_{absMAX} along the entire length of the absorbent core 104. The first and the second fastening wing 109,110 are asymmetrical with respect to any transverse axis extending in the transverse axis and with respect to each other, as seen along a longitudinal axis extending in a longitudinal direction L. The first and the second fastening wings 109,110 are furthermore symmetrically disposed with respect to each other along a diagonal axis extending in a direction D in the plane of the sanitary article and with an angle α within the range of from 10° to 65° with respect to the longitudinal centerline C_{L} of the sanitary article 100, such as for example within the range of from 30° to 55°. Hence, as may be seen in this FIG. 1, the first fastening wings 109 may be a mirror-image of the second fastening wing 110 if the first fastening wings 109 is turned 180° and be arranged to extend outboard at the respective first and second longitudinal side edge 105,106 at a respective location along the respective first and second longitudinal side edge 105,106 such that they are symmetrically disposed with respect to each other.

The first fastening wing 109 has a width w_{f1} continuously increasing over at least 75 of the first fastening wing 9, as seen from the rear edge 108 and towards the front edge 107 of the sanitary article 100. The second fastening wing 110 has a width w_{f2} continuously increasing over at least 75% of the second fastening wing 100, as seen from the front edge 107 and towards the rear edge 108 of the sanitary article 100. The sum of the first and the second wing maximum widths w_{fm1}+w_{fm2} is greater than a width w_{absMID} of the absorbent core 4 as measured at the sanitary article transverse centerline C_{T}, however due to the asymmetry of the fastening wings 109,110, the fastening wings 109,110 may be folded narrowly about the longitudinal edges 105,106 of the sanitary articles 100 in a non-overlapping configuration.

The first fastening wing 109 has a first fastening wing maximum width w_{fm1} and the second fastening wing 110 has a second fastening wing maximum width w_{fm2}. The first fastening wing maximum width w_{fm1} is measured from the first longitudinal absorbent core edge 113 and at the widest section of the first fastening wing 109 as seen in the transverse direction T. The second fastening wing maximum width w_{fm2} is measured from the second longitudinal absorbent core edge 114 and at the widest section of the second fastening wings 110 as seen in the transverse direction T. The sum of the first and the second fastening wing maximum widths w_{fm1}+w_{fm2} is at least 10 mm greater than the maximum absorbent core width w_{absMAX}. The sum of the first and the second fastening wing widths w_{fm1}+w_{fm2} is furthermore at least 10 mm greater than the absorbent core width w_{absMID}, as measured at a transverse centerline C_{T} of the sanitary article 100. In this FIG. 1, the sanitary article 100 is illustrated in an unfolded configuration.

The topsheet may include or consist of fibrous nonwoven layer(s) being spunbonded, meltblown, carded, hydroentangled, wetlaid. Suitable nonwoven materials can be composed of natural fibers, such as woodpulp or cotton fibers, synthetic thermoplastic fibers, such as polyolefins, polyesters, polyamides and blends and combinations thereof or from mixtures of natural and synthetic fibers. The materials suited as topshet material should be soft and non-irritating to the skin and be readily penetrated by body fluid, such as menstrual fluid and urine.

The absorbent core may be of any conventional kind. Examples on commonly used absorbent materials are cellulosic fluff pulp, tissue layers, highly absorbent polymers (co-called superabsorbents), absorbent foam materials, absorbent nonwoven materials or the like. It is common to combine cellulosic fluff pulp with superabsorbents in an absorbent structure. It is also common to have absorbent structures comprising layers of different material with different properties with respect to liquid acquisition capacity, liquid distribution capacity and storage capacity.

The backsheet may consist of a thin plastic film, e.g. a polyethylene or polypropylene film, a nonwoven material coated with a liquid impervious material, a hydrophobic nonwoven, which resist liquid penetration. Laminates of plastic films and nonwoven materials may also be used. The backsheet material can be breathable to allow vapor to escape from the absorbent structure, while still preventing liquids from passing through the backsheet material.

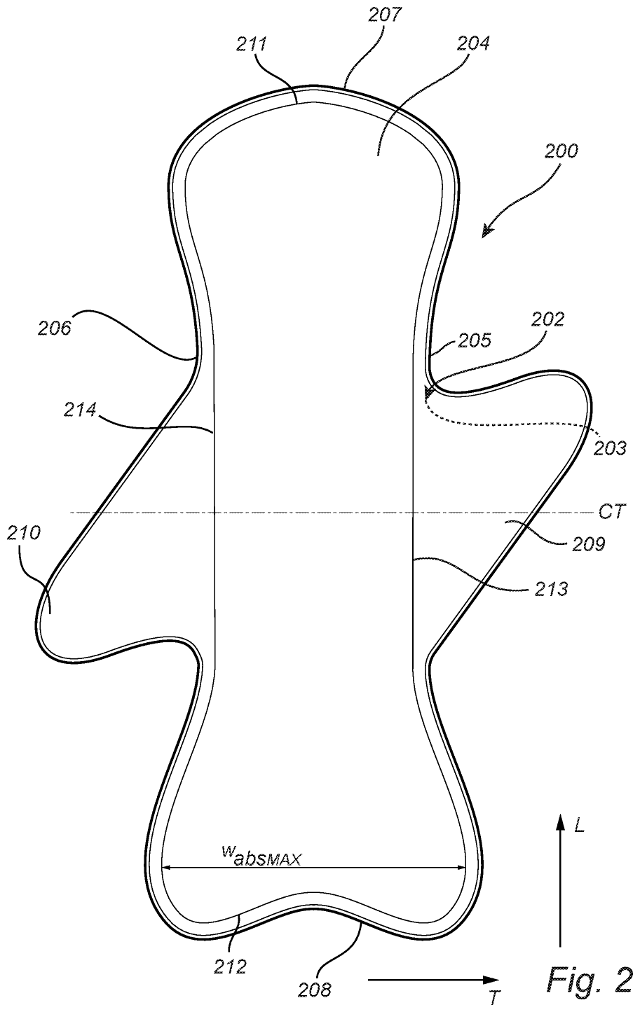

FIG. 2 illustrates a sanitary article 200 according to the present disclosure. The sanitary article 200 comprising a topsheet 202, a backsheet 203 and an absorbent core 204 positioned between the topsheet 202 and the backsheet 203. The sanitary article 200 has a longitudinal axis extending in a longitudinal direction L and a transverse axis extending in a transverse direction T. The sanitary article 200 has a first longitudinal side edge 205, a second longitudinal side edge 206, a front edge 207 and a rear edge 208. The sanitary article 200 has a first fastening wing 209 extending at the first longitudinal side edge 205 and a second fastening wing 210 extending at the second longitudinal side edge 206. The first and the second fastening wings 209,210 are asymmetrical with respect to any transverse axis and with respect to each other, as seen along the longitudinal axis and with respect to a longitudinal centerline of the sanitary article. The absorbent core 204 comprises an absorbent core front edge 211, an absorbent core rear edge 212 and absorbent core first and second longitudinal edges 213,214. The sanitary article 200 is illustrated in an unfolded configuration. However, when the sanitary article 200 is folded, it has a folded article width and the folded article width is maximum 10 mm greater than the maximum absorbent core width w_{absMAX}. The folded article width being taken in the transverse direction T and at the widest section of the folded article. The folded sanitary article width is the width of the folded sanitary article as packaged. Hence if only the fastening wings are folded and the sanitary article is provided without transverse folding in a package, then the width is measured at the widest section along the entire length of the sanitary article. If the article is folded over one, two or three transverse fold lines, the width is measured at the final folded article.

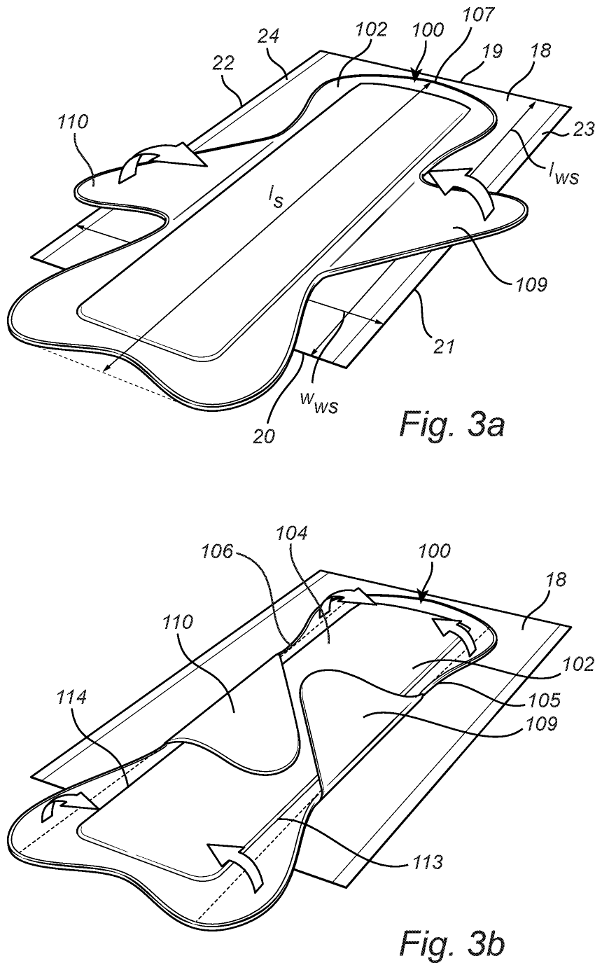

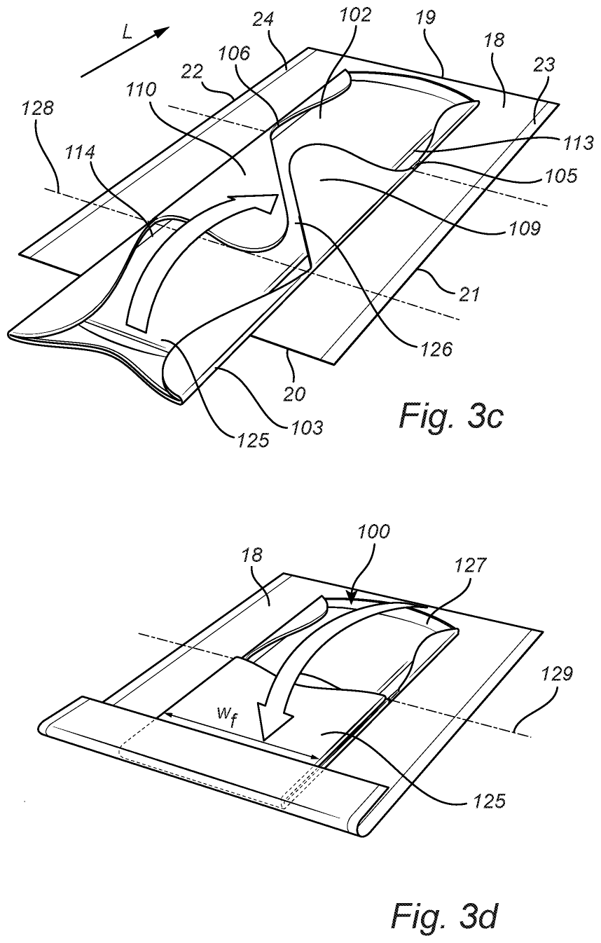

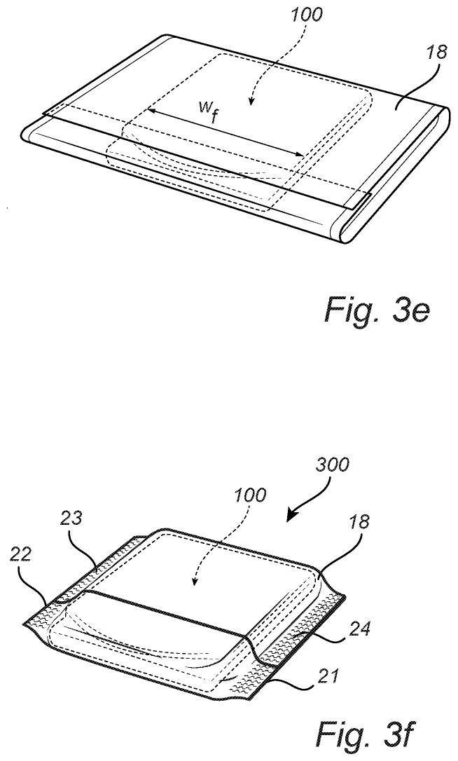

FIGS. 3a-3f illustrate folding of the sanitary article 100 according to FIG. 1 and the wrapping of the folded sanitary article in a wrapping sheet 18 to provide an individually packaged sanitary article 300, shown in FIGS. 3e and 3f. However, the sanitary article 200 according to FIG. 2 may be folded in the same manner as illustrated in FIGS. 3a-3f. The wrapping sheet 18 has a first transverse sheet edge 19 and a second transverse sheet edge 20. The wrapping sheet 18 has a first longitudinal sheet edge 21 and a second longitudinal sheet edge 22. The wrapping sheet 18 has a sheet width w_{ws} extending between the first sheet longitudinal edges 21 and the second sheet longitudinal edges 22. The wrapping sheet 18 comprises a first longitudinal sealing area 23 and a second longitudinal sealing area 24 sealing the respective first and second longitudinal sheet edges 21,22 of the wrapping sheet 18. The length of the sanitary article 100 is greater than a wrapping sheet length l_{ws}, which is from 75% to 90% of the length lₛ of the sanitary article 100. The length lₛ of the sanitary article 100 corresponds to the greatest length of the sanitary article 100, meaning that if the front edge 107 and/or the rear edge 108 are irregular as in this figure a respective transverse line is drawn at the frontmost point of the front edge and at the rearmost point of the rear edge and the length is measured therebetween.

In a first step illustrated in FIG. 3a, the first and the second fastening wings 109,110 are folded onto of the topsheet 102 of the sanitary article 100. FIG. 3b illustrates the sanitary article 100 with the first and the second fastening wings 109,110 being folded on top of the topsheet 102. The first fastening wing 109 is arranged close to, such as from 0 mm to 6 mm, the second fastening wing 110 when the fastening wings 109,110 are in a folded, non-overlapping, position. When the absorbent core 104 is essentially rectangular while a width of the sanitary article 100 varies along the length of the sanitary article 100, such as if the sanitary article has an hourglass shape, a portion along the first and second longitudinal side edge 105,106 may be folded on top of the topsheet 102, essentially along the longitudinal side edges 113,114 of the absorbent core 104, such that the folded sanitary article has an essentially rectangular shape, as illustrated in FIG. 3c. As the folded longitudinal edge portions only comprise topsheet and backsheet material, the portion is very flexible and easy to fold.

In FIG. 3c, a distance between the first longitudinal absorbent core edge 113 and the first longitudinal sealing area 23 of the wrapping sheet 18 and a distance between the second longitudinal absorbent core edge 114 and the second longitudinal sealing area 24 of the wrapping sheet 18 each range from 10 mm to 25 mm, as seen along the entire length of the absorbent core 104. A distance between the first longitudinal absorbent core edge 113 and the first longitudinal sheet edge 21 and a distance between the second longitudinal absorbent core edge 114 and the second longitudinal sheet edge 22 each range from 10 mm to 35 mm. However, the distance may be from 0 mm, such as if the wrapping sheet is open along the longitudinal side edges. The distance may for example also be up to 50 mm.

In FIG. 3c, a rear portion 125 of the sanitary article 100, together with the wrapping sheet 18, is subsequently folded over central portion 126 of the sanitary article 100, about a first fold line 128 and on top of the folded fastening wings 109,110. The sanitary article 100 is provided with adhesive on the backsheet 103 thereof and may either be releasably attached to the wrapping sheet 18 directly or be releasably attached to a release paper which may be attached, such as permanently attached, to the wrapping sheet 18.

In a subsequent step illustrated in a step 3d, a front part 127 of the sanitary article 100 is folded over the rear folded portion 125 of the sanitary article 100 about a second fold line 129 such that the sanitary article 100 is completely wrapped with the wrapping sheet 18, as shown in FIG. 3e.

When the sanitary article 100 is folded, as seen in FIG. 3e, it has a folded article width wf and the folded article width wf is maximum 10 mm greater than the maximum absorbent core width w_{absMAX} (see FIG. 3e). The folded article width being taken in the transverse direction T and at the widest section of the folded article. The folded sanitary article width is the width of the final folded sanitary article as packaged. Hence if only the fastening wings are folded and the sanitary article is provided without transverse folding in a packages, then the width is measured at the widest section along the entire length of the sanitary article. If the article is folded over one, two or three transverse fold lines, the width is measured at the final folded article.

FIG. 3f illustrates the wrapping sheet 18 sealed around the sanitary article 100. The wrapping sheet 18 has been sealed along the first and second longitudinal sealing areas 23,24 to close the package along the longitudinal sides 21,22 of the wrapping sheet 18 and form an individually package of a folded sanitary article 100. The longitudinal sides 21,22 of the wrapping sheet 18 may for example be adhesive sealed or ultrasonically sealed.

The sanitary article according to the present disclosure may thus be provided with an adhesive, such as a pressure-sensitive adhesive on the backsheet. The elongated wrapping sheet may be releasably adhered the adhesive-bearing backsheet of the sanitary article. The wrapping sheet may be coated with material which enables the release of the wrapping sheet, such as silicone, so that the wrapping sheet may be peeled away from the adhesive-bearing backsheet of the sanitary article and so that the sanitary article may be attached to an undergarment. The adhesive-bearing region of the backsheet may also be covered with a release paper. The release paper may be attached to the wrapping sheet, for example with a permanent attachment, such that the sanitary article is attached to the wrapping sheet via the release paper.

1. A folded sanitary article, such as a sanitary napkin or a pantiliner, the folded sanitary article comprising a topsheet, a backsheet and an absorbent core arranged between the topsheet and the backsheet, the sanitary article extending in a longitudinal direction (L) and in a transverse direction (T), the sanitary article having a first longitudinal side edge, a second longitudinal side edge, a front edge and a rear edge, the sanitary article having a first fastening wing extending outboard at the first longitudinal side edge and a second fastening wing extending outboard at the second longitudinal side edge, the absorbent core comprising an absorbent core front edge, an absorbent core rear edge, an absorbent core first longitudinal side edge and an absorbent core second longitudinal side edge, the absorbent core having an absorbent core width (w_{abs}) and a maximum absorbent core width (w_{absMAX}), the first and the second fastening wings of the sanitary article being folded onto the topsheet of the sanitary article, the folded sanitary article having a folded article width (w_{f}), wherein the first and second fastening wings are asymmetrical with respect to each other, as seen along the longitudinal axis and that the folded article width (w_{f}) is not more than 10 mm greater than the maximum absorbent core width (w_{absMAX}).

2. The folded sanitary article according to claim 1, wherein the first and second fastening wings are asymmetrical with respect to any transverse axis extending in the transverse direction (T).

3. The folded sanitary article according to claim 1, wherein the first and the second fastening wings are symmetrically disposed with respect to each other along a diagonal axis extending in a direction (D), in the plane of the sanitary article and with an angle α within the range of from 10° to 65° with respect to a longitudinal centerline (C_{L}) of the sanitary article, optionally with an angle α within the range of from 30° to 55° with respect to a longitudinal centerline (C_{L}) of the sanitary article.

4. The folded sanitary article according to claim 1, wherein the first fastening wing has a first wing edge section and the second fastening wing has a second fastening wing edge section, the first and second fastening wing edge sections being arranged opposite and adjacent each other when being folded onto the topsheet of the sanitary article and a distance (d) between the first wing edge section and the second wing edge section is from 0 to 25 mm, optionally the distance (d) between the first wing edge section and the second wing edge section is from 0 to 10 mm.

5. The folded sanitary article according to claim 1, wherein the first fastening wing has a first fastening wing maximum width (w_{fm1}) and the second fastening wing has a second fastening wing maximum width (w_{fm2}), each of the fastening wing maximum widths (w_{fm1}, w_{fm2}) being measured from the respective first and a second longitudinal edge of the absorbent core and at the widest section of the respective fastening wing as seen in the transverse direction (T), and wherein the sum of the fastening wing maximum widths (w_{fm1}+w_{fm2}) is more than 10 mm greater than the absorbent core width (w_{absMID}), as measured at a transverse centerline (C_{T}) of the sanitary article.

6. The folded sanitary article according to claim 1, wherein the folded sanitary article is a tri-folded sanitary article, being folded about a first and a second transverse folding line.

7. The folded sanitary article according to claim 6, wherein the absorbent core has an essentially rectangular or square folded shape.

8. The folded sanitary article according to claim 6, wherein the absorbent core has an essentially rectangular shape having an absorbent core width (w_{abs}) not varying more than ±20 mm along a length of the absorbent core.

9. The folded sanitary article according to claim 6, wherein the absorbent core has an essentially rectangular shape having an absorbent core width (w_{abs}) not varying more than ±10 mm along a length of the absorbent core.

10. The folded sanitary article according to claim 6, wherein the folded sanitary article is individually packed in a wrapping sheet, the wrapping sheet having first and second transverse sheet edges and first and second longitudinal sheet edges, a sheet width (w_{ws}) extending between the first and the second sheet longitudinal edge.

11. The folded sanitary article (100) according to claim 10, wherein the wrapping sheet (18) comprising a first and a second longitudinal sealing area sealing the respective first and second longitudinal edges of the wrapping sheet.

12. The folded sanitary article according to claim 11, wherein a distance between the first longitudinal absorbent core edge and the first longitudinal sealing area of the wrapping sheet and a distance between the second longitudinal absorbent core edge and the second longitudinal sealing area of the wrapping sheet each ranges from 0 mm to 50 mm, as seen along the entire length of the absorbent core, optionally each ranges from 5 mm to 25 mm, as seen along the entire length of the absorbent core.

13. The folded sanitary article according to claim 10, wherein the sheet width (w_{ws}) is not more than 60% greater than the width (w_{absMID}) of the absorbent core, as measured at the transverse centerline (C_{T}), optionally the sheet width (w_{ws}) is not more than 40% greater than the width (w_{absMID}) of the absorbent core, as measured at the transverse centerline (C_{T}).

14. The folded sanitary article according to claim 10, wherein a distance between the first longitudinal absorbent core edge and the first longitudinal sheet edge and a distance between the second longitudinal absorbent core edge and the second longitudinal sheet edge each ranges from 0 mm to 35 mm, optionally each ranges from 5 mm to 30 mm.

15. The folded sanitary article according to claim 10, wherein a total surface area of the wrapping sheet is maximum 150% greater than a total surface area of the absorbent core.

16. The folded sanitary article according to claim 10, wherein the total surface area of the wrapping sheet is maximum 120% greater than the total surface area of the absorbent core.

17. The folded sanitary article according to claim 10, wherein the sanitary article is wrapped by a wrapping sheet having a wrapping sheet length (l_{ws}) being from 75% to 135% of a length (lₛ) of the sanitary article.

18. The folded sanitary article according to claim 10, wherein the wrapping sheet length (l_{ws}) is from 75% to 100% of the length (lₛ) of the sanitary article.

19. A package including a plurality of folded sanitary articles, such as a sanitary napkins or a pantiliners, each of the folded sanitary articles comprising a topsheet, a backsheet and an absorbent core arranged between the topsheet and the backsheet, each of the sanitary articles extending in a longitudinal direction (L) and in a transverse direction (T), each of the sanitary articles having a first longitudinal side edge, a second longitudinal side edge, a front edge and a rear edge, each of the sanitary articles having a first fastening wing extending outboard at the first longitudinal side edge and a second fastening wing extending outboard at the second longitudinal side edge, the absorbent core comprising an absorbent core front edge, an absorbent core rear edge, an absorbent core first longitudinal side edge and an absorbent core second longitudinal side edge, the absorbent core having an absorbent core width (wabs) and a maximum absorbent core width (wabsMAX), the first and the second fastening wings of each of the sanitary articles being folded onto the topsheet thereof, each of the folded sanitary articles having a folded article width (wf), wherein the first and second fastening wings are asymmetrical with respect to each other, as seen along the longitudinal axis and that the folded article width (wf) is not more than 10 mm greater than the maximum absorbent core width (wabsMAX) .

20. An array of folded sanitary articles , such as a sanitary napkins or a pantiliners, wherein each of the folded sanitary articles includes a topsheet, a backsheet and an absorbent core arranged between the topsheet and the backsheet, each of the sanitary articles extending in a longitudinal direction (L) and in a transverse direction (T), each of the sanitary articles having a first longitudinal side edge, a second longitudinal side edge, a front edge and a rear edge, each of the sanitary articles having a first fastening wing extending outboard at the first longitudinal side edge and a second fastening wing extending outboard at the second longitudinal side edge, the absorbent core comprising an absorbent core front edge, an absorbent core rear edge, an absorbent core first longitudinal side edge and an absorbent core second longitudinal side edge, the absorbent core having an absorbent core width (wabs) and a maximum absorbent core width (wabsMAX), the first and the second fastening wings of each of the sanitary articles being folded onto the topsheet thereof, each of the folded sanitary articles having a folded article width (wf), wherein the first and second fastening wings are asymmetrical with respect to each other, as seen along the longitudinal axis and that the folded article width (wf) is not more than 10 mm greater than the maximum absorbent core width (wabsMAX) , the array comprising a first folded sanitary article the folded sanitary articles and a second folded sanitary article of the folded sanitary articles, wherein the first folded sanitary article has a first sanitary article length (lₛ₁) and the second folded sanitary article has a second folded sanitary article length (lₛ₂) and wherein the a first sanitary article length (lₛ₁) is at least 20% longer than the second folded sanitary article length (lₛ₂).
